(19) United States
(12) Patent Application Publication
(10) Pub. No.: US 2008/0103563 A1
(43) Pub. Date: May 1, 2008

(54) LIGHT THERAPY PERSONAL CARE DEVICE

(75) Inventors: Steven D. Powell, Orem, UT (US); Andrew Michael Smith, Orem, UT (US)

Correspondence Address: STOEL RIVES LLP - SLC, 201 SOUTH MAIN STREET, ONE UTAH CENTER, SALT LAKE CITY, UT, 84111, US

(73) Assignee: LUMIPORT, LLC, Provo, UT (US)

(21) Appl. No.: 11/679,425

(22) Filed: Feb. 27, 2007

Related U.S. Application Data
(60) Provisional application No. 60/863,085, filed on Oct. 26, 2006.

(51) Int. Cl. A61N 5/067

(52) U.S. Cl. 607/89

(57) ABSTRACT

A personal care device comprising a phototherapy component and a microdermabrasion component is disclosed. The phototherapy device component may comprise an array of light emitting diodes (LEDs), which are configured to emit light over a range of wavelengths which are selected to treat a skin condition. The LED array may be disposed behind the exfoliating surface of the microdermabrasion component so light therapy treatment and microdermabrasion treatment may be performed simultaneously.

### CROSS-REFERENCE TO RELATED APPLICATIONS

This application claims the benefit under 35 U.S.C. §119(e) of U.S. Provisional Patent Application Ser. No. 60/863,085, filed Oct. 26, 2006, and titled “Light Therapy Personal Care Device,” which is incorporated herein by reference.

### TECHNICAL FIELD

The present disclosure relates generally to phototherapy devices. More specifically, the present disclosure relates to the integration of a phototherapy device and a microdermabrasion device.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is an exploded perspective view of one embodiment of a combined phototherapy and microdermabrasion device used in the treatment of skin conditions.

FIG. 2 is a plan view of the combined phototherapy and microdermabrasion device of FIG. 1.

FIG. 3A is a top plan view of one embodiment of a removable microdermabrasion head for use with a phototherapy device for the treatment of skin conditions.

FIG. 3B is a side elevation view of the removable microdermabrasion head of FIG. 3A.

FIG. 3C is a bottom plan view of the removable microdermabrasion head of FIG. 3A.

FIG. 4 is a plan view of a control panel of one embodiment of a combination phototherapy and microdermabrasion device.

FIG. 5 is a side elevation view of a recharging base station and a combination phototherapy and microdermabrasion device absent the microdermabrasion head.

FIG. 6A is a front plan view of one embodiment of a combination phototherapy and microdermabrasion device absent the microdermabrasion head.

FIG. 6B is a side elevation view of the combination phototherapy and microdermabrasion device of FIG. 6A.

FIG. 7 is a perspective view of one embodiment of a combination rotary shaver and phototherapy device.

FIG. 8 is a perspective view of another embodiment of a combination foil shaver and phototherapy device.

FIG. 9 is a block diagram of a system for treating various skin conditions with a phototherapy component of a personal care device.

FIG. 10 is a front plan view of another embodiment of a combination phototherapy and microdermabrasion device.

### DETAILED DESCRIPTION

It will be readily understood that the components of the embodiments as generally described and illustrated in the Figures herein could be arranged and designed in a wide variety of different configurations. In some cases, well-known structures, materials, or operations are not shown or described in detail. The following more detailed description of various embodiments, as represented in the Figures, is not intended to limit the scope of the present disclosure, but is merely representative of various embodiments. While the various aspects of the embodiments are presented in drawings, the drawings are not necessarily drawn to scale unless specifically indicated.

For this application, the phrases “connected to” and “coupled to” refer to any form of interaction between two or more entities, including mechanical, electrical, magnetic, electromagnetic, fluid, and thermal interaction. Two components may be coupled to each other even though they are not in direct contact with each other.

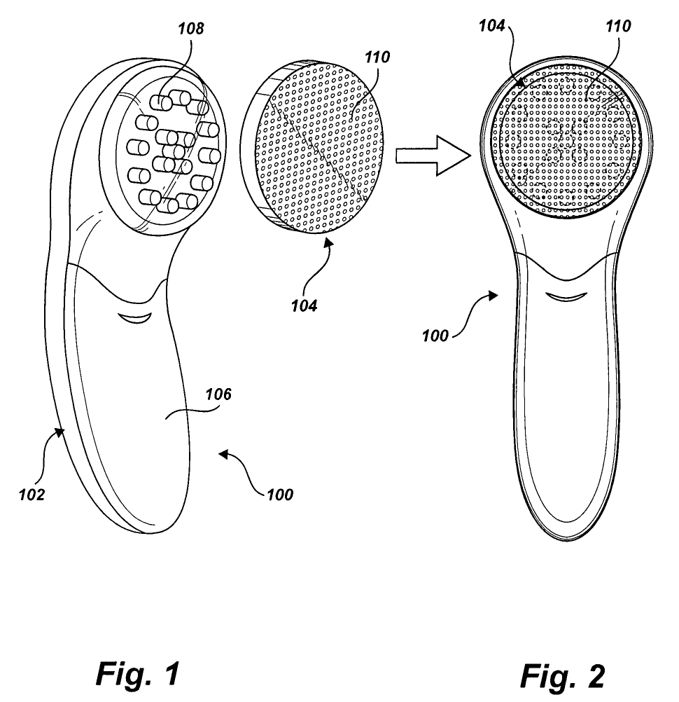

FIG. 1 represents one embodiment of a combined microdermabrasion and phototherapy device 100 used in the treatment of various skin conditions, as shown from an exploded perspective view. Device 100 may comprise a phototherapy component 102 and a microdermabrasion component 104. The phototherapy component 102 may include a housing 106 that can be easily gripped by a user. An array of light emitting diodes (“LEDs”) 108 is disposed at one end of component 102 to provide light therapy treatment to a user in need thereof. Alternatively, different light sources may be used such as, but not limited to, lasers, incandescent lamps, fluorescent lamps, plasma arc lamps, and the like. For example, the phototherapy array 108 may comprise laser light, at a single or multiple wavelengths.

The microdermabrasion component 104 may be a removable microdermabrasion head 104 that is coupled to the phototherapy component 102 over the LED array 108. The microdermabrasion component 104 may be used to provide a microdermabrasion treatment to a user separately or simultaneously with the light therapy treatment.

In one embodiment, the microdermabrasion head 104 may snap on to the phototherapy component 102 through a deflectable portion (not shown) of the microdermabrasion head 104. Various other methods and structures for coupling the microdermabrasion head 104 to the phototherapy component 102 may be used as would be appreciated by one having skill in the art, such as an interference fit, a threaded coupling, a clip mechanism, and the like.

Once the phototherapy component 102 is activated, the LEDs 108 emit at least one wavelength of light. As would be appreciated by those having skill in the art, a “single wavelength” may actually represent a very narrow range of wavelengths centered around and having a maximum at the single wavelength. LEDs 108 may be desirable since they typically use less power, produce less heat, and have a longer life span than most incandescent lamps. Furthermore, LEDs 108 are often an inexpensive alternative to wavelength selection compared to lamp and filter systems.

In one embodiment, the LEDs 108 may all emit the same narrow range of wavelengths and the light emitted may be considered monochromatic. In alternative embodiments, multiple LED types may be used to emit various wavelengths. In yet other embodiments, multi-color LEDs may be used to emit more than one discrete wavelength. For example, the multi-color LEDs 108 may comprise bi-color, or bi-polar LEDs producing two discrete wavelengths. In other embodiments, the multi-color LEDs 108 are tri-color LEDs producing three discrete wavelengths. As would be apparent to those having skill in the art, multi-color LEDs 108 may be used which can produce more than three discrete wavelengths as the advancement of technology permits.

By way of example, in one embodiment, the LED array 108 may produce a narrow band of wavelengths in the red portion of the visible electromagnetic spectrum as well as a narrow band of wavelengths in the blue portion of the visible electromagnetic spectrum, using either an array of monochromatic LEDs or multi-color LEDs. The red wavelengths may range between about 630 nanometers and about 680 nanometers, while the blue wavelengths may range between about 400 nanometers and about 470 nanometers. In one embodiment, the red band is between about 650 to about 670 nanometers and the blue band is between about 405 to about 420 nanometers. Moreover, the LED array 108 may be capable of producing just red wavelengths at one time, or just blue wavelengths, or both red and blue wavelengths simultaneously.

While device 100 functions to provide light therapy to the skin of a user, the device 100 may simultaneously provide microdermabrasion therapy treatment. However device 100 may separately administer light therapy treatment or microdermabrasion treatment as desired by the user. Microdermabrasion therapy is an exfoliation technique used to remove the top layers of skin on a user's face or body. This may be accomplished by having a coarse surface or pad, similar to sandpaper, on the front face 110 of the microdermabrasion head 104. In some embodiments, microdermabrasion may be used to lessen the appearance of facial wrinkles and improve the aging appearance of the skin.

The phototherapy component 102 may also be used to treat a variety of skin conditions. The LED array 108 of device 100 may be directed toward or placed on a region of skin having a particular skin condition so that the skin may be treated with light therapy. The phototherapy component 102 may produce specific wavelengths to treat a number of skin conditions. For example, for the treatment of acne both blue wavelengths (about 400 to about 470 nanometers) and red wavelengths (about 630 to about 680 nanometers) may be used. Furthermore, for the treatment of acne, the phototherapy component 102 may provide twice as much exposure to blue wavelengths than to red wavelengths in a single treatment event. Relative exposures of red and blue wavelengths may be determined through a quantifiable value such as light intensity or duration of exposure.

In order to treat wrinkles or other signs of aging in the skin, blue, red and yellow wavelength bands may be used. The blue and red wavelengths may range between about 400 to about 470 nanometers and about 630 to about 680 nanometers, respectively. The yellow band of wavelengths may be between about 530 nanometers and about 600 nanometers. Red and yellow wavelengths may also be used in treatment of scarring.

In treating rosacea and other forms of redness, a yellow range of wavelengths may be used between about 530 and about 600 nanometers. Red and blue wavelengths could also be used in various embodiments.

In treating sun spots, a yellow range of wavelengths (about 530 to about 600 nanometers) may be used. For alternative forms of sun damage, a red band (about 630 to about 680 nanometers) may be employed.

Blue light (between about 400 and about 470 nanometers) may be used to treat and kill bacteria that may cause various forms of skin blemishes, such as acne.

Inflammation may be treated by exposing affected skin to red wavelengths (about 630 to about 680 nanometers) and also to infrared wavelengths, which may range from about 800 nanometers to about 1000 nanometers.

Lesions in the skin may be treated by illuminating the affected area with red wavelengths (about 630 to about 680 nanometers) and infrared wavelengths (about 800 to about 1000 nanometers).

Skin blemishes may be treated through exposure to red, blue and yellow wavelengths. As discussed above the wavelength ranges may be about 630 to about 680 nanometers for red, about 400 to about 470 nanometers for blue, and about 530 to about 600 nanometers for yellow.

Razor burn, hyperpigmentation and pseudofolliculitis barbae may be treated through exposure to red, yellow, and an extended range of infrared wavelengths. As discussed above the wavelength ranges may be about 630 to about 680 nanometers for red and about 530 to about 600 nanometers for yellow. The extended range for infrared wavelengths may be from about 800 to about 1300 nanometers.

Additionally, to the extent that hyperpigmentation and pseudofolliculitis barbae is exacerbated or caused by the presence of bacteria, a blue range of wavelengths may be applied, from about 400 to about 460 nanometers.

LEDs 108 that emit a band of wavelengths in the green portion of the visible electromagnetic spectrum may also be used in treating sun spots, rosacea and wrinkles. The wavelength range associated with green light may range between about 500 nanometers and about 530 nanometers. LED light therapy may also be used in treating dead skin and other skin problems.

FIG. 2 depicts the combined microdermabrasion and phototherapy device 100, as shown from a plan view. The microdermabrasion head 104 is coupled to the phototherapy component 102 as described in conjunction with FIG. 1. In one embodiment, the front face 110 may be translucent to allow for the transmission of light from the LED array 108 disposed behind the microdermabrasion head 104. The front face 110 may be comprised of a thin translucent, transparent or semi-transparent layer, such as a plastic film. Alternatively a translucent foam may be used.

In yet another alternative embodiment, the microdermabrasion head 104 may include orifices that extend through the front face 110 and correspond spatially with the underlying LEDs 108. In this embodiment, the microdermabrasion head 104 may not need to have a translucent surface since each orifice would allow light to pass there through and treat the skin while simultaneously providing a microdermabrasion treatment.

The front face 110 of the microdermabrasion head 104 may have a rough surface capable of performing an exfoliation function. In some embodiments, the exfoliation pad located on the front face 110 may be replaceable. The microdermabrasion head 104 may vibrate, oscillate or spin to remove damaged, dead or older skin cells through exfoliation. In one embodiment, the microdermabrasion head vibrates or oscillates separately from the body 106 of the phototherapy component 102. In another embodiment, the body portion 106 of device 100 vibrates along with the microdermabrasion head 104.

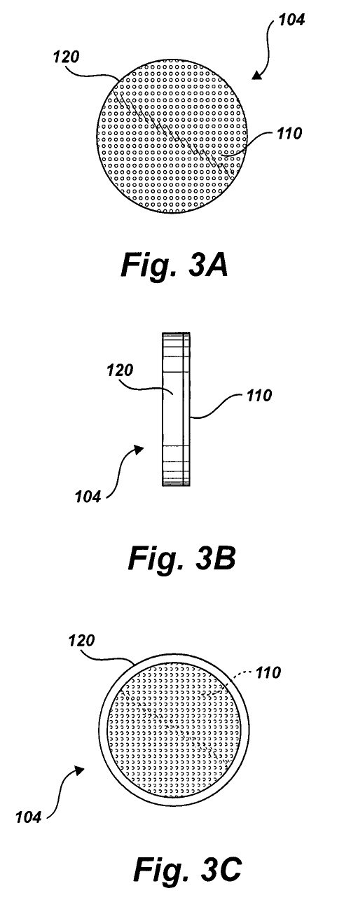

FIGS. 3A, 3B and 3C depict one embodiment of a removable microdermabrasion head 104 for use with a phototherapy device for the treatment of skin conditions, as shown from a top plan view, a side elevation view and a bottom plan view, respectively. The removable microdermabrasion head 104 may include an exfoliating pad on its front face 110, which is coupled to an annular collar 120. The annular collar 120 may be coupled to the phototherapy component through a deflectable tab (not shown) or an interference fit as disclosed herein.

The exfoliating pad may be removable and/or disposable after a period of use. Furthermore, as discussed above, the front face 110 may be translucent, transparent, or semi-transparent to allow for the transmission of light there through. Alternatively, the front face 110 may be opaque, but have orifices that correspond with the position of each individual LED in the LED array. Having the LED array situated behind the front face 110 of the microdermabrasion head provides for the conservation of space, allows for a more compact device and provides better coverage of light against the skin when being used simultaneously during a microdermabrasion treatment.

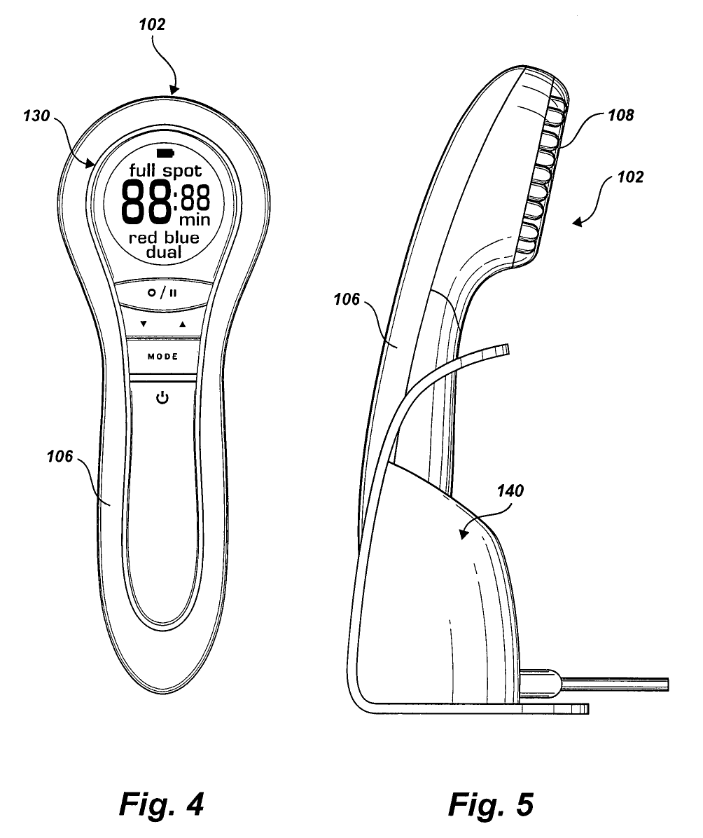

Referring to FIG. 4, a control panel 130 is shown on the backside of the phototherapy component 102 of device 100. The control panel 130 may control the duration of light therapy and/or microdermabrasion treatment. The control panel 130 may also set the conditions of light and/or microdermabrasion treatment. For example, control panel 130 may select which wavelengths are used in a particular treatment, i.e., blue, red, yellow, green, ultraviolet, infrared, and combinations thereof as discussed herein. Furthermore, in some embodiments, the control panel 130 may be programmed to emit a combination of wavelengths simultaneously to treat different skin conditions at the same time. Control panel 130 may also control the intensity of the light emitted from the LED array. The intensities of each color may also be varied independently in some embodiments.

Programming for the control panel 130 may be built-in and/or user-configurable. For example, the control panel 130 may include built-in parameters for wavelength, time, and intensity for treating a number of skin conditions. The user may be allowed to modify those parameters for his or her unique skin types and/or conditions. In certain embodiments, the control panel 130 may include a communications port, such as a universal serial bus (USB) port, for interfacing the device 100 to a computer (not shown). Using software on the computer, the user may be able to modify any of the built-in parameters or even download parameters (including complete treatment regimens for different skin conditions) from a website. In one implementation, the website may allow customized treatment regimens to be created and stored for different users, which could be downloaded to the device 100 as needed.

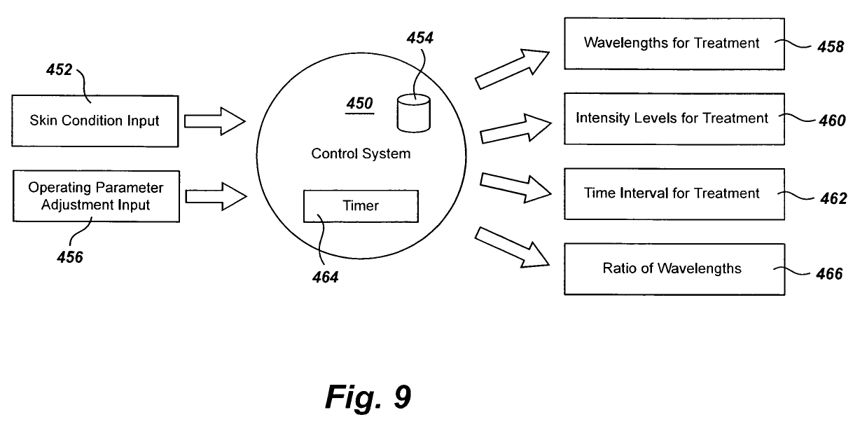

Control panel 130 may also control and select the amplitude of the vibrations/oscillations of the microdermabrasion head when being used in a microdermabrasion treatment mode. Other features of the control panel 130 may include a power button, LED on/off button, an interrupt button, a battery power display, timer, wavelength selection and/or other alternative displays. For example, a LCD screen may optionally prompt a user for input or indicate operating status, etc. One embodiment of a control system associated with the control panel 130 of device 100 is described in greater detail in conjunction with FIG. 9.

In one embodiment, the LEDs 108 are illuminated only when the microdermabrasion component 104, or some other portion of the device 100, is in contact with (or in close proximity to) the user's skin. For example, a pressure sensor may be placed beneath the microdermabrasion component 104 in order to sense when the device 100 is pressed against the user's skin. In other embodiments, touch sensors, such as TouchCells™, manufactured by TouchSensor Technologies, LLC, may be used. When voltage is applied to the touch sensor, an electric field is created. The field emanates through any dielectric substrate such as glass or plastic. When a conductive mass enters the field, the sensor detects the change and indicates an event has occurred. The input stimulus to the field can take the form of contact with the user's skin. Various other proximity sensors and/or photoelectric sensors, as known in the art, could also be used.

FIG. 5 depicts the phototherapy component 102 of device 100 from a side elevation view within a docking station 140. Docking station 140 may comprise a recharging base station. Device 100 may be powered by an internal portable power source, such as a battery. The battery power source may provide device 100 with sufficient power that AC power is not required.

When phototherapy component 102 is cradled within docking station 140 as depicted, the docking station 140 may have contact points that are in electronic communication with contact points of the phototherapy device 102. The docking station 140 is also connected to an AC power supply through a power cord. Alternatively, the phototherapy device 102 may be recharged using an AC adapter.

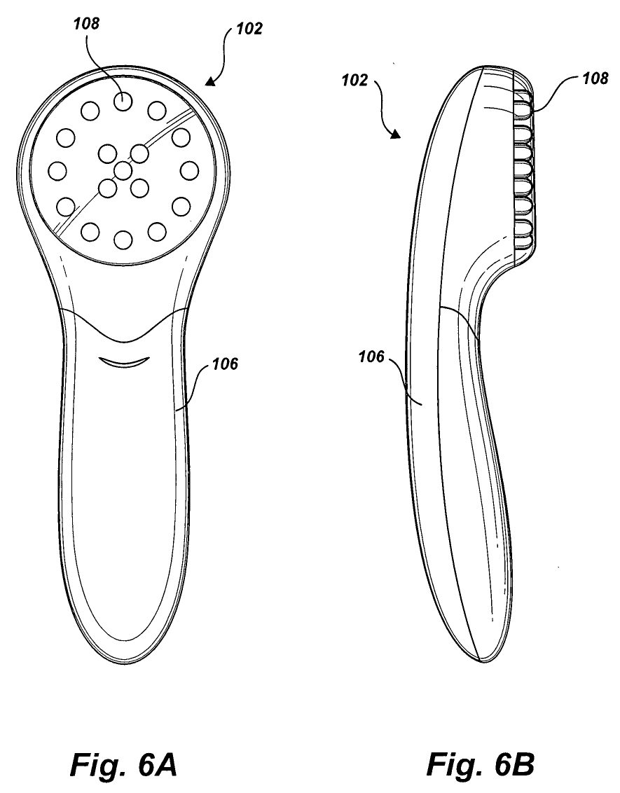

FIGS. 6A and 6B represent the phototherapy component 102 of device 100 absent the microdermabrasion head, as shown from a front plan view and side elevation view, respectively. The housing 106 of the phototherapy component 102 may be ergonomically shaped so a user can easily grip it. The LED array 108 may also be disposed at one end of component 102 to provide light therapy treatment to a user in need thereof. In some embodiments, the LED array 108 may have a cover or lens which is transparent to visible light, but functions to diffuse ultra violet light or other harmful rays that may inadvertently be emitted from the phototherapy device 102.

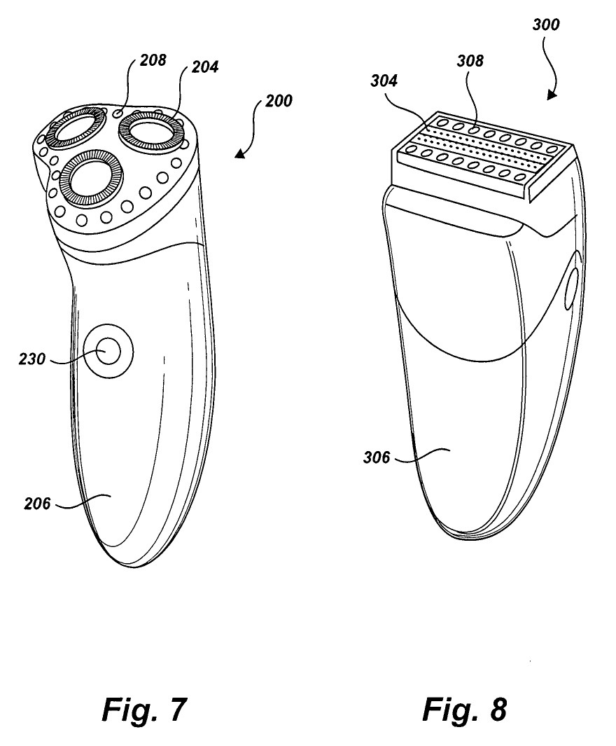

Referring to FIG. 7, a rotary shaver phototherapy device 200 is depicted from a perspective view. Device 200 may comprise a shaver having rotary shaving heads 204 for shaving hair on a user's face or other portion of the body. Device 200 includes a body portion 206 that may be easily gripped by a user. A LED array 208 may be disposed adjacent the rotary shaving heads 204 to provide light therapy treatment to a user in need thereof.

The LED array 208 may provide light therapy treatment simultaneously with the daily process of shaving. Having the LED array 208 disposed adjacent the rotary shaving heads 204 provides better coverage of light against the skin when being used simultaneously during a shave. Alternatively, rotary shaving heads 204 may be used without activation of the LED array 208 depending on the phototherapy treatment regimen.

The rotary shaver phototherapy device 200 may include a power button or switch 230 for activation of the rotary shaving heads 204, the LED array 208 or both. Additionally, device 200 may include a control panel (not shown) similar to the control panel discussed in conjunction with FIG. 4, optionally with the pressure sensor/touch sensors as described. Device 200 may also be coupled to a recharging base station similar to the docking station described in conjunction with FIG. 5.

As discussed in connection with the combined microdermabrasion phototherapy device, the LED array 208 of device 200 may be capable of transmitting various wavelengths of light for a particular treatment, e.g., blue, red, yellow, green, ultraviolet, infrared, and combinations thereof as discussed above. Moreover, monochromatic or multi-chromatic LEDs may be used as described herein. Furthermore, the various features described in connection with the embodiments of FIGS. 1 through 6B may be incorporated herein, as appropriate.

FIG. 8 depicts a foil shaver phototherapy device 300. Device 300 may comprise a shaver having foils 304 for shaving hair on a user's face or other portion of the body. Device 300 may include an ergonomic body portion 306 that may be easily gripped by the user. Adjacent the foils 304 is situated a LED array 308 (or alternative light source, such as laser light) to provide light therapy treatment to a user in need thereof.

As with the rotary shaver embodiment described in FIG. 7, the LED array 308 of device 300 may provide light therapy treatment simultaneously with the daily process of shaving. Having the LED array 308 disposed adjacent the foils 304 provides better coverage of light against the skin when being used simultaneously during a shave. Alternatively, foils 304 may be used without activation of the LED array 308 depending on the phototherapy treatment regimen.

Device 300 may include a control panel (not shown) similar to the control panel discussed in conjunction with FIG. 4, optionally with the pressure sensor/touch sensors as described. Device 300 may also be coupled to a recharging base station similar to the docking station described in conjunction with FIG. 5. Additionally, the foil shaver phototherapy device 300 may transmit various wavelengths of light for a particular skin treatment regimen, e.g., blue, red, yellow, green, ultraviolet, infrared, and combinations thereof as discussed above. Moreover, monochromatic or multi-chromatic LEDs may be used as described herein. Furthermore, the various features described in connection with the embodiments of FIGS. 1 through 6B may be incorporated herein, as appropriate.

Other types of shaving devices may also be adapted to include an LED array 308. For instance, a disposable razor head of the type illustrated in U.S. Pat. No. 5,630,275 may be equipped with an LED array 308. Power may be supplied from a battery within the razor head, itself, or the non-disposable handle portion.

Referring collectively to FIGS. 8 and 9, the shaver phototherapy device 200, 300 may comprise an assembly that is modular in nature. The shaver phototherapy assembly 200, 300 may comprise a phototherapy device module including a light source configured to emit a wavelength of light selected to treat a skin condition. The assembly 200, 300 may also comprise a shaving device module including a shaving head configured to shave hair from the body surface of the user. The phototherapy device module and the shaving device module may be packaged together.

In one embodiment, the phototherapy device module is separate from the shaving device module, but may be used together in a shaving/photo therapy treatment regimen. In another embodiment, the phototherapy device module and the shaving device module may be coupled together to conduct a shaving/photo therapy treatment regimen. In yet another embodiment, the phototherapy device module and the shaving device module may be integrated into a single device.

FIG. 9 is a block diagram of a control system 450 for treating various skin conditions with a phototherapy component of a personal care device, such as 100, 200, 300. The control system 450 may be incorporated, in part, into a control panel as heretofore described. The control system 450 may receive various forms of user input in order to control various treatment modes of the phototherapy device.

For example, a user may provide input 452 indicative of a skin condition that a user desires to be treated by the device. Examples of various skin condition inputs 452 may include acne, rosacea, wrinkles, inflammation, sun spots or sun damage, bacteria, blemishes, lesions, razor burn, hyperpigmentation or pseudofolliculitis barbae. A user may select one or more of a list of skin conditions to be treated and the control system 450 accesses operating parameters stored on a memory device 454 or database in machine readable form. The operating parameters of the phototherapy device that correspond with a particular light therapy treatment may be inputted by a manufacturer or programmer of the device, or alternatively a user may provide adjustment operating parameter input 456 in accordance with a customized LED skin treatment program.

The control system 450 accesses the memory device 454 containing multiple operating parameters and selects those corresponding to the skin condition input 452 received. The phototherapy component of the device then runs according to the operating parameters corresponding with the selected skin condition input 452. One example of an operating parameter output of the control system 450 is a control signal corresponding to the specific wavelengths for treatment 458 of the skin condition selected. Accordingly, if acne is selected by the user, the control system 450 accesses the corresponding operating parameter that indicates both red and blue wavelengths are to be used for treatment. However, if the user selected rosacea as the skin condition to be treated, the wavelengths for treatment 458 may be in the yellow band (about 530 to about 600 nanometers).

Another form of output of the control system 450 is the operating parameter that indicates the intensity levels 460 for treatment of the skin condition selected. For example, in one embodiment the intensity levels of a LED may be 105 mW/cm². However, an exemplary alternative intensity level output 460 of 92 mW/cm² may be provided by the control system 450. A user may adjust the intensity level output 460 corresponding to a particular skin treatment. The user adjusts that particular operating parameter through input 456 indicating an increase or a decrease in intensity to treat more severe or less severe skin conditions, respectively. Intensity adjustments may be made, for example, in percentage increments such as ±5%, ±10%, ±15%, etc.

Another operating parameter that may be controlled is the time interval for treatment 462. An exemplary treatment session may last 15 minutes for some skin conditions. However, treatment session times may be less, such as between 3 and 15 minutes, depending upon the user input. The time interval for treatment 462 may be controlled by a timer 464, which may be embodied, for example, as a Real Time Clock (RTC). Once the skin condition input 452 is received and the corresponding operating parameters accessed, the indicated time interval 462 is controlled by the timer 464. Once the timer 464 reaches the time interval 462 indicated it automatically shuts off LED emission.

Additionally, the operating parameters corresponding to a skin condition input 452 may include wavelength ratio data 466. For example, when acne is selected as the skin condition to be treated, the operating parameters corresponding with the treatment of acne may indicate that twice as much exposure to blue wavelengths as compared to red wavelengths is desired. Consequently, the wavelength ratio 466 for acne would be 2:1, blue to red. The relative exposures of red and blue wavelengths may be determined through a quantifiable value such as light intensity or duration of exposure. Therefore, blue LED light may be emitted at twice the intensity of red LED light. Alternatively, the exposure time of blue LED light during a particular treatment interval would be twice as long as red LED light. This may be accomplished by pulsating blue LEDs twice as much as red LEDs, or by activating twice as many blue LEDs than red LEDs, or other methods known to those having skill in the art. The 2:1 ratio as disclosed is representative of one embodiment, and as would be appreciated by those having skill in the art alternative wavelength ratios 466 may be used.

Accordingly, a user is able to control the wavelengths emitted, the intensity levels, the time intervals for treatment, and the relative ratio of wavelengths produced by simply selecting a particular skin condition. By selecting the skin condition, the control system 450 causes the LED phototherapy device to provide the appropriate colors, intensity, etc., for that skin condition.

The control system may be in electronic communication with a control panel, such as that discussed in conjunction with the description of FIG. 4. By way of example, the control panel may include a LCD display which may show an indication of the skin condition selected by the user and the associated operating parameters. In some embodiments, the display may show a countdown of time left or time elapsed for the particular light therapy treatment. Furthermore, an audible alert, such as a beep, may let the user know when the treatment event has ended.

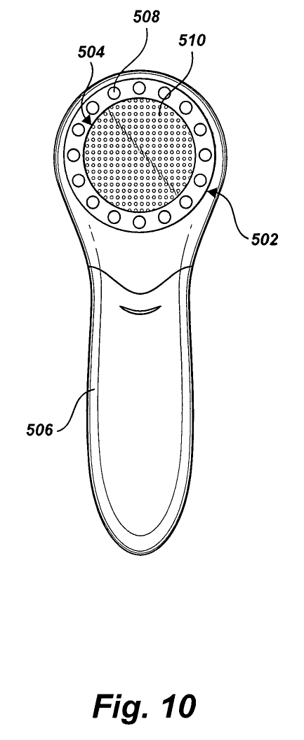

FIG. 10 depicts another embodiment of a combined microdermabrasion and phototherapy device, as shown from a front plan view. The microdermabrasion head 504 may be coupled adjacent to the phototherapy component 502. In one embodiment, the microdermabrasion head 504 is removable, such that new or different heads having different surfaces may be used. In alternative embodiments the microdermabrasion head 504 is permanently affixed adjacent the phototherapy component 502.

In the embodiment depicted in FIG. 10, the phototherapy component 502 comprises an LED array 508 that surrounds the microdermabrasion head 504. In alternative embodiments, additional LEDs may be disposed behind the front face 510 of the microdermabrasion head 504, which is optionally translucent or transparent as described in conjunction with FIGS. 1 through 3C.

The front face 510 of the microdermabrasion head 504 may have a rough surface capable of performing an exfoliation function. The microdermabrasion head 504 may vibrate, oscillate or spin to remove damaged, dead or older skin cells through exfoliation. In one embodiment, the microdermabrasion head vibrates or oscillates separately from the body 506 of the phototherapy component 502. In another embodiment, the body portion 506 vibrates along with the microdermabrasion head 504.

While specific embodiments and applications of phototherapy personal care devices have been illustrated and described, it is to be understood that the disclosure is not limited to the precise configuration and components provided. Various modifications, changes, and variations apparent to those of skill in the art may be made in the arrangement, operation, and details of the devices and systems disclosed.

What is claimed is:

1. A handheld personal care device, comprising:
a phototherapy device component comprising an array of light sources configured to emit light at at least one wavelength selected to treat a skin condition; and
a microdermabrasion device component comprising an exfoliating surface configured to exfoliate a user's skin;
wherein the array of light sources is disposed adjacent the exfoliating surface, such that light therapy treatment and microdermabrasion treatment may be performed simultaneously.

2. The device of claim 1, wherein the array of light sources comprises an array of light emitting diodes (LEDs).

3. The device of claim 2, wherein at least one LED of the array of LEDs comprises a multi-color LED capable of emitting more than one discrete wavelength.

4. The device of claim 2, wherein the LED array emits light at a wavelength in a blue portion of the visible electromagnetic spectrum and light at a wavelength in a red portion of the visible electromagnetic spectrum.

5. The device of claim 4, wherein the blue wavelength is between 400 nanometers and 470 nanometers and the red wavelength is between 630 nanometers and 680 nanometers.

6. The device of claim 1, wherein the array of light sources is disposed behind the exfoliating surface of the microdermabrasion device component.

7. The device of claim 6, wherein the exfoliating surface is translucent such that light from the array of light sources diffusively passes through the exfoliating surface.

8. The device of claim 7, wherein the exfoliating surface is a translucent foam.

9. The device of claim 6, wherein the exfoliating surface is transparent such that light from the array of light sources passes through the exfoliating surface.

10. The device of claim 6, wherein the exfoliating surface comprises orifices which correspond spatially with each light source of the array of light sources, such that light may be transmitted beyond the exfoliating surface via the orifices.

11. The device of claim 1, wherein the array of light sources is disposed around the outer perimeter of the exfoliating surface.

12. The device of claim 1, wherein the microdermabrasion device component comprises an exfoliating head which is removably coupled to the personal care device.

13. The device of claim 1, further comprising a control system to control the array of light sources according to operating parameters, the operating parameters including at least one of: intensity level of light source emission, duration of light source emission, and wavelength selection.

14. The device of claim 1, wherein the skin condition is acne.

15. The device of claim 1, wherein the skin condition is rosacea.

16. The device of claim 1, wherein the skin condition is wrinkles.

17. The device of claim 1, wherein the skin condition is inflammation.

18. The device of claim 1, wherein the skin condition is sun damage.

19. The device of claim 1, wherein the skin condition is caused by bacteria.

20. The device of claim 1, wherein the skin condition is at least one of: blemishes, lesions, and scarring.

21. The device of claim 1, wherein the skin condition is razor burn.

22. The device of claim 1, wherein the skin condition is hyperpigmentation.

23. The device of claim 1, wherein the skin condition is pseudofolliculitis barbae.

24. The device of claim 1, wherein the array of light sources comprises at least one laser.

25. The device of claim 1, further comprising a portable power source.

26. A handheld personal care device, comprising:
a phototherapy device component comprising one or more light sources configured to emit light over a number of wavelengths selected to treat a skin condition;
a microdermabrasion device component configured to exfoliate a user's skin; and
a control system to control the one or more light sources according to operating parameters, the operating parameters including at least one of: intensity level of light emission, duration of light emission, and wavelength selection;
wherein light therapy treatment and microdermabrasion treatment may be performed simultaneously, and wherein the phototherapy device component, microdermabrasion device component, and control system are integrated into a portable unit.

27. The device of claim 26, wherein the skin condition is selected from at least one of: acne, rosacea, wrinkles, inflammation, sun damage, bacteria, blemishes, lesions, razor burn, hyperpigmentation and pseudofolliculitis barbae.

28. The device of claim 26, wherein the one or more light sources comprise at least one multi-color LED capable of emitting more than one discrete wavelength.

29. The device of claim 26, wherein the one or more light sources comprise at least one laser.

30. A portable personal care device, comprising:
a light emitting diode (LED) illumination source configured to emit at least one wavelength selected to treat a skin condition;
a microdermabrasion head comprising an exfoliating surface configured to exfoliate a user's skin, wherein the LED illumination source is disposed behind the exfoliating surface, such that light therapy treatment and microdermabrasion treatment may be performed simultaneously;
a machine readable medium for storing operating parameters of the LED illumination source, the operating parameters corresponding to treatment of skin conditions; and
a control system to receive input from the user indicative of a skin condition to be treated, such that the control system accesses the operating parameters corresponding to the indicated skin condition and the control system controls the LED illumination source in accordance with the corresponding operating parameters;
wherein the operating parameters include at least one wavelength for treatment of each skin condition.

31. The device of claim 30, wherein the operating parameters further include at least one intensity level of the light produced by the LED illumination source for treatment of each skin condition.

32. The device of claim 30, wherein the operating parameters further include at least one time interval representing a length of time the LED illumination source emits light for treatment of each skin condition.

33. The device of claim 32, wherein the control system comprises a timer which is set according to the at least one time interval of the operating parameters corresponding to the indicated skin condition, such that emission of the LED illumination source is automatically discontinued when the at least one time interval has elapsed.

34. The device of claim 30, wherein the operating parameters further include at least one wavelength ratio representing how much of a quantifiable value of one wavelength is emitted relative to the quantifiable value of another wavelength.

35. The device of claim 30, wherein the operating parameters are adjustable by a user.

36. The device of claim 30, further comprising a display in electronic communication with the control system, the display showing the skin condition inputted by the user.

37. The device of claim 30, further comprising a display in electronic communication with the control system, the display showing a timer indicating a treatment time.

38. The device of claim 30, further comprising a portable power source.
